(19) United States
(12) Patent Application Publication
(10) Pub. No.: US 2006/0216494 A1
(43) Pub. Date: Sep. 28, 2006

(54) Organic-inorganic nanocomposite coatings for implant materials and methods of preparation thereof

(76) Inventors: Helga Furedi-Milhofer, Jerusalem (IL); Pazit Bar Yosef Ofir, Jerusalem (IL); Maja Dutour Sikiric, Zadar (CR); Csilla Gergely, Montpellier (FR); Frederic Cuisinier, Montpellier (FR)

Correspondence Address: Kevin D McCarthy;Roach Brown McCarthy & Gruber, 1620 Liberty Building, Buffalo, NY, 14202, US

(21) Appl. No.: 10/535,939

(22) PCT Filed: Nov. 18, 2003
(86) PCT No.: PCT/IL03/00975

§ 371 Date: May 8, 2006

Related U.S. Application Data
(60) Provisional application No. 60/428,725, filed on Nov. 25, 2002.

(51) Int. Cl. B32B 5/16; A61F 2/28

(52) U.S. Cl. 428/323; 428/330; 427/2.1; 427/402; 427/430.1; 623/23.57

(57) ABSTRACT

The present invention provides inorganic-organic nanocomposite coatings for implant materials and methods for the production thereof. The coatings consist of a sequentially adsorbed polyelectrolyte film (SAPF) intergrown with calcium phosphate crystals. The substrate is selected from glass, polymer, metal or metal alloys. The SAPFs consist of successions of positively and negatively charged monolayers, comprising biocompatible polyelectrolytes, preferably polyaminoacids. The calcium phosphate crystals may comprise octacalcium phosphate, calcium deficient apatites, carbonate apatites, hydroxyapatite, or mixtures thereof, with particle sizes 50 nm to 2 μm. The inorganic phase is grown “in situ” within the polyelectrolyte organic matrix.

### FIELD OF THE INVENTION

The invention generally relates to organic-inorganic nano-composite coatings for implant materials, mainly referring to orthopedic and dental implants, and to methods of preparation thereof.

### BACKGROUND OF THE INVENTION

While most metals and metal alloys meet many of the biomechanical requirements of load bearing implants, they are bioinert or biotolerant and thus show poor or nonexistent interfacial bonding between the metallic surface and the surrounding bone. To alleviate this problem, different surface coatings consisting of calcium phosphates have been applied. Coating methods previously employed with some success include plasma spraying, which gives tight adhesion between hydroxyapatite and the metal plate. Drawbacks of this method are that it requires costly equipment and high processing temperatures. The high temperatures employed cause significant structural alterations in the coatings, which may result in mechanical failure at the interface metal-coating interface and within the coating itself.

More recently processes for obtaining hydroxyapatite coatings by direct precipitation onto the implant material from solutions containing calcium and phosphate ions and/or various foreign ions (including magnesium, carbonate, or other) have been proposed. In U.S. Pat. No. 5,188,670 assigned to Brent, a complicated process and apparatus for coating porous substrates with hydroxyapatite film has been described. Essentially, the method comprises combining calcium and phosphate solutions of relatively high concentrations, at elevated temperatures between 60 to 90° C., to obtain hydroxyapatite crystals, which are then, in a specially designed apparatus, precipitated onto the surface to be coated. Coating methods disclosed in U.S. Pat. No. 6,280,789 assigned to Rey et al., and further in U.S. Pat. No. 6,207,218, assigned to Layrolle et al. are simpler, in both procedures the material to be coated, e.g., a medical implant was submerged in an aqueous solution containing calcium, phosphate and bicarbonate ions and spontaneous precipitation of carbonated apatite was initiated in the presence of the implant by raising the supersaturation in situ. The supersaturation was regulated by either raising the temperature, and thus the pH, by removing some of the carbonate or by bubbling alternately CO₂ or air through the solution. A drawback of these methods is that the coatings are simply precipitated onto the substrate surface but are in no way anchored to it. They are thus likely to be unstable and not likely to withstand rough implanting procedures. A related procedure, described in the art is based on soaking a metal substrate for two weeks in very dilute solutions, containing calcium, phosphate and other inorganic ions, which would produce a calcium phosphate coating. Optionally one or more biologically active, organic substances could be co-precipitated. This method seems to suffer from the same problems as above, which the authors were trying to overcome by adjusting the surface roughness of the substrate and using prolonged coating times, thus inducing slow growth from very dilute solutions. Consequently, the method is rather time consuming and the deposits are ill defined in terms of composition and structure. The coatings showed cracking and fractures and their appearance was dependent on both the material and the surface of the metal substrates used.

A new approach of producing calcium phosphate coatings, presented by Bunker et al., Science 264, 1994, 48, calls for modifying substrate surfaces by introducing functional groups, which should mediate the deposition of calcium phosphate mineral under mild conditions. The idea is based on the observation that in nature organisms use various macromolecules, containing different functional groups, i.e. carboxylic, sulfate and phosphate groups, to induce and control mineralization. Accordingly, it was assumed, that on functionalized surfaces mineralization would readily proceed from relatively dilute solutions at low temperatures and under mild conditions (close to physiological). Such methods should be cost-effective and adaptable to a variety of ceramic, polymeric and metallic materials. Various methods to introduce functional groups into different substrates have been proposed.

Many investigators, such as Kokubo and collaborators (See Acta Mater. 46, 1998, 2519; Materials Science Forum 293, 1999, 65 for example) tried to introduce functional groups to various substrates, such as bioglass, glass ceramics and titanium metal surfaces. The methods of treatment depended on the specific substrate, to which coating was to be applied. Titanium plates where soaked for 24 h in concentrated NaOH solutions and subsequently heated to a temperature between 500 to 600° C. Coatings were then deposited by soaking the plates for several days in a so-called simulated body fluid, SBF, i.e. a solution of ionic concentrations similar to those in blood plasma. Samples thus treated showed relatively high bonding strengths, in comparison to bioglass and glass ceramics, between the coating and the metal surface. It was further shown that titanium plates functionalized with Ti—OH and Ti—OOH groups specifically induce oriented crystallization of hydroxyapatite and octacalcium phosphate (OCP).

However, the coatings described were not well defined in terms of composition and structure and were nor evenly spread over the coated surface. Also, the proposed methods are rather time and energy consuming.

To enhance the speed of deposition and the thickness of the coatings, the inventors of U.S. Pat. No. 6,129,928 to Sarangapani et al. proposed to covalently bind a nucleating agent with acidic functional groups to the surface hydroxyl groups of titanium plates. In addition, post-treatment with diluted hydrogels is proposed, to reinforce the inorganic structure and enhance the mechanical strength of the coating. Growth factors and other reactive proteins can be included, by coupling them to the hydrogel molecules. Although this patent presents a significant improvement over previous art, the method is substrate specific, as it presupposes a substrate with reactive surface hydroxyl groups, to which a nucleating agent can be covalently bound.

Finally, US Pat. No. 2002/018798 to Dard et al. discloses coatings, comprising an organic-inorganic composite system, which consists of a collagen matrix mineralized with calcium phosphate. The collagen matrix is prepared by immersing the substrate into a solution of collagen type I, which is then reconstituted by adjusting the pH and temperature. The collagen fibrils thus obtained are mineralized by an electrochemical method, in which the coated substrate serves as one of the electrodes. Thus, since the substrate has to be conductive, the method is restricted to metals. Also, although the material is similar to bone tissue, it does not contain acidic functional groups, which are thought to be responsible for biological mineralization.

Most recently, US Pat. No. 2002/0037383, assigned to Spillman et al. disclosed a method to enhance the biocompatibility of medical devices by introducing electrostatically self-assembled thin film coatings. Hwang et al. sequentially covered orthopedic metals with polylysine and polyglutamic acid, and then exposed the surface to organoapatite-precipitating solutions comprising polylysine (Hwang et al.: J. Biomed. Mater. Res. 47, 1999, 504).

The present invention is based on experience known in the art with polyelectrolyte multilayer films, as well as with the crystallization of calcium phosphates and their interactions in solution with polyelectrolytes and extracellular matrix proteins. It has been shown in the art that it is possible to fabricate polyelectrolyte multilayer films on substrates by consecutive adsorption of polyanions and polycations or other charged molecular or colloidal objects. Such films are mainly dependent on the properties of the chosen polyelectrolytes and much less on the underlying substrate or the substrate charge density. It has also been demonstrated in the art that nucleation and growth of calcium phosphate crystals in neutral or basic aqueous solutions is induced by an amorphous precursors phase, and the crystal morphology is specifically influenced by polyelectrolytes, such as polyaminoacids and matrix proteins, which may be present in solution. A method for changing the surface free energy, based on multilayer film, was shown to increase the nucleation activity of surfaces. In order to combine the high nucleation activity of calcium phosphate crystals and of polyelectrolyte and thus enhance the bioactivity of orthopedic and dental implants, we have developed methods for embedding calcium phosphate, adsorbed and/or grown “in situ” on polyelectrolyte multilayer films.

### SUMMARY OF THE NVETION

It is thus an object of the present invention to provide an organic-inorganic multilayer nanocomposite composition, comprising a plurality of organic polyelectrolyte films, and an intergrowth of nanometer to micron-sized inorganic crystalline bioactive particles, grown “in situ” within the organic matrix. The aforementioned polyelectrolytes are preferably selected from the group of polyaminoacids, such as poly-leucine, poly-arginine, poly-lysine, poly-glutamic acid, poly-serine, poly-aspartic acid, poly-hydroxyproline; polypeptides or proteins such as collagen, gelatine, elastin, amelogenin, albumin, sialoprotein, osteocalcin, phosphoproteins, specifically phosphophoryn, fibrinogen, fibronectin; polysaccharides such as lipopolysacharide, dextrin, cyclodextrin, heparin, chitosan, hyaluronic acid, agarose, alginate glucosaminoglycan, heparan, chondroitin, chondroitin sulfate, proteoglycan; or they may comprise other biocompatible polymers and/or small molecules, such as poly(lactide), polyinosinic acid, polycytidylic acid, polythymidylic acid, polyguanylic, polystyrene sulfonate, poly(acrylic) acid, poly(methacrylic) acid, poly (ethylene glycol), poly(galacturonic) acid, poly(maleimide), silk, phosvitin, polyphosphonates, polyphosphates, lectines, lactic acid, glycolic acid, glycin, their derivatives or any mixture thereof. The hereto-defined bioactive inorganic crystals preferably comprise crystalline calcium phosphates grown in situ within the organic matrix. More specifically, the aforementioned crystalline calcium phosphates comprise calcium hydrogen phosphate, octacalcium phosphate, tri-calcium phosphate, calcium deficient apatite, carbonated apatite, stoichiometric hydroxyapatite, crystalline calcium phosphates containing foreign ions, crystalline calcium phosphates containing cytokines, crystalline calcium phosphates containing peptides, their derivatives or any combination thereof.

It is also in the scope of the present invention to provide a most effective bioactive nanocomposite coating comprising the composition as defined in any of the above. Moreover, it is further in the scope of the present invention to provide implants, comprising the aforementioned compositions. More specifically, hereto-defined implants are at least partially coated by the aforementioned compositions, in the manner that a significant portion of said implants are coated by a bioactive nanocomposite. Those implants preferably comprise materials selected from composite materials, glass ceramics, polymer, metal, metal alloys, or any combination thereof. The said metal or metal alloy are at least partially made of titanium, titanium based alloys, stainless steel, tantalum, zirconium, nickel, tantalum, iridium, niobium, palladium, nickel-titanium, alloys based thereon or any combination thereof.

It is further in the scope of the present invention to provide a method comprising inter alia the steps of adsorbing polyelectrolytes on top of a surface so that at least one film is obtained; washing the obtained film in the manner that residual polyelectrolytes are removed; depositing nano-sized to micron sized particles of ACP on top of said polyelectrolyte film, so that at least one film comprising calcium-containing, bioactive inorganic material is formed; washing the obtained film in the manner that residual calcium containing solution is removed; and then immersing the material into a metastable calcifying solution in the manner that “in situ” growth of crystalline calcium phosphate is induced and sustained.

It is also in the scope of the present invention to provide a method comprising inter alia the steps of adsorbing polyelectrolytes on top of a surface so that at least one film is obtained; washing the obtained film in the manner that residual polyelectrolytes are removed; depositing nano-sized to micron-sized particles of ACP on top of said polyelectrolyte film, so that at least one film comprising calcium-containing bioactive inorganic material is formed; washing the obtained calcified film in the manner that residual calcium containing solution is removed; adsorbing polyelectrolytes on top of said calcium phosphate layer; wherein said sequence of steps is repeated in the manner that calcified SAPF is obtained; and then immersing the obtained material into a metastable calcifying solution in the manner that in situ growth of calcium phosphate crystals is induced and sustained within the calcified SAPF.

### BRIEF DESCRIPTION OF THE DRAWINGS

In order to understand the invention and to see how it may be implemented in practice, a plurality of preferred embodiments will now be described, by way of non-limiting example only, with reference to the accompanying figures, in which

FIGS. 1a, 1b and 1c are representing data recorded by the OWLS technique for the build-up of SAPF, (PLL/PGA)₅PLL (a); (PLL/PGA)₆ (b) and (c) from MES/TRIS buffer (a) and (b) and from HEPES buffer (c), and the adsorption of ACP from water [(a) and (b)] and from HEPES buffer (c), respectively;

FIG. 2 is representing SEM micrographs, in two different magnifications, of aggregated ACP particles deposited on glass, coated with (PLL/PGA)₁₅;

FIG. 3 is showing SEM micrographs of ACP particles in material A, in two magnifications;

FIG. 4 is showing SEM micrographs of ACP particles in material A and B, side view;

FIG. 5 is showing SEM micrographs of coating C, two different magnifications;

FIG. 6 is showing SEM micrographs of (a) coating C, and (b) coating D, side views;

FIG. 7 is showing surface morphologies of coatings C (a-b) and D (c-f) before and after the adhesive tape test;

FIG. 8 relates to coating D+, FIG. 8a is a micrograph of the surface morphology, FIG. 8b is an EDX spectrum, and FIG. 8c is a thin layer XRD spectrum in which characteristic peaks of apatite are mark arrows.

FIG. 9 is showing adhesion and proliferation of human osteoblast cells onto bare titanium (L1), OCP deposited on bare titanium (L2), titanium coated with (PLL/PGA)₁₀ (L3), titanium coated with (PLL/PGA)₁₀-apatite-(PLL/PGA)₅ (coating C+; L5), titanium coated with (PLL/PGA)₁₀-apatite-(PLL/PGA)₅-apatite-(PLL/PGA)₅ (coating D+; L7) and plastic (golden standard. L8).

### DETAILED DESCRIPTION OF THE INVENTION

The following description is provided, alongside all chapters of the present invention, so as to enable any person skilled in the art to make use of said invention and sets forth the best modes contemplated by the inventors of carrying out this invention. Various modifications, however, will remain apparent to those skilled in the art, since the generic principles of the present invention have been defined specifically to provide bioactive organic-inorganic nanocomposite coatings for implant materials and to methods of preparation thereof.

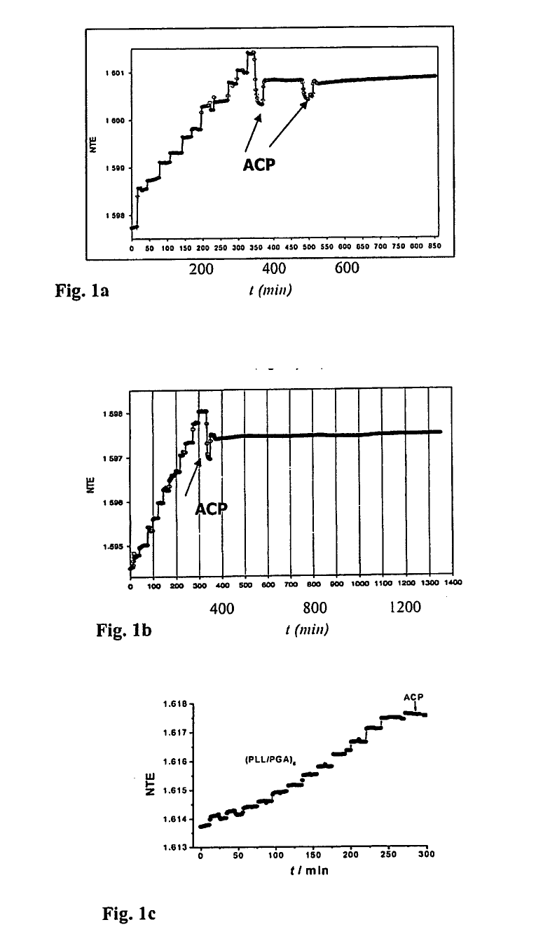

The present invention generally relates to organic-inorganic composite coatings, comprising sequentially adsorbed polyelectrolyte films (i.e., SAPF), intergrown with nanometer to micrometer sized inorganic crystals. The SAPFs are constructed as previously described by Decher, G. Science 277 (1997) 1232, by consecutively adsorbing positively and/or negatively charged polyions from their respective solutions, Adsorbed between the organic layers are particles of an inorganic precursor phase from which crystal growth is initiated and sustained by immersion of the material into a metastable solution, supersaturated to the desired inorganic crystals (see FIG. 1).

For the purpose of the present invention, organic polyelectrolytes are selected in a non-limiting manner from biocompatible or at least partially biocompatible polyelectrolytes, such as polyaminoacids, proteins, polysaccharides, polyphosphonates, polyphosphates, phosphoproteins, and any other synthetic or natural biocompatible or partially biocompatible polymers and/or mixtures of the same etc., all hereto denoted in the term organic ‘PE’.

Hence, it is in the scope of the present invention wherein a plurality of polycation compositions is sequentially adsorbed on top of a plurality of polyanion PE films or vice versa. It is further in the scope of the present invention wherein a polycation or polyanion composition is sequentially adsorbed on top of another polycation or polyanion layer, respectively. It is still in the scope of the present invention wherein nonionic compositions are utilized inter alia in said sequentially adsorbed PE films.

Further according to the present invention, the term ‘SAPF’ is referring to any film comprising sequentially adsorbed PE films, e.g., a multilayer matrix or a multi-stratum matrix, a conglomerated matrix, a crystallized matrix, amorphous structures, vesicular or sponge like structures or any combination thereof.

Moreover, the term ‘film’ generally relates according to the present invention to any homogeneous or heterogeneous, continuous or discontinuous, isotropic or anisotropic bioactive films, coatings or layers, at least partially comprising SAPF as defined in any of the above.

The term ‘bioactive’ is generally referring to bioactive calcium-containing compositions, composites and devices. It is acknowledged in this respect that bioinert portions provided in those compositions are also possible. The materials according to the present invention can also be biodegradable in the manner that it is either dissolved or resorbed in the body. It is according to yet another embodiment of the present invention, wherein the term ‘bioactive’ is also referring to any at least partially biocompatible compositions, composites and devices.

Intergrown within the organic matrix are nanometer to micrometer sized calcium phosphate crystals, or other inorganic particles. It is well in the scope of the present invention wherein the aforementioned calcium phosphate particles, or other inorganic particles have inorganic polyelectrolyte characteristics. It is also in the scope of the present invention wherein the inorganic bioactive particles comprise crystalline calcium phosphates, such as calcium hydrogenphosphate, octacalcium phosphate, tricalcium phosphate, calcium deficient apatite, carbonated apatite, stoichiometric hydroxyapatite with specific properties or mixtures of some of the above, grown directly on and/or within the organic matrix. It is yet acknowledged in this respect that other inorganic bioactive crystals of different compositions and structures are possible.

According to the present invention, the calcium phosphate is grown directly on and/or within the organic film during the building up period. Hence, the preparation of calcium phosphate layers is based on the adsorption or embedding of amorphous calcium phosphate particles, hereto defined in the term ‘ACP’ and/or another suitable precursor phase into the SAPF and subsequent growth of crystalline octacalcium phosphate or calcium deficient hydroxyapatite from a metastable supersaturated solution, henceforth calcifying solution, crystal growth being induced and mediated by the precursor particles and/or the adsorbed polyelectrolyte layers. The SAPF-calcium phosphate assembly is formed by the following sequence of steps:
i. adsorbing a sufficient amount of organic PE onto a predetermined substrate surface;
ii. cleansing said upper layer of said substrate at least partially coated by said organic composition by means of removing the residual polyelectrolyte(s) by washing;
iii. depositing ACP and/or another suitable precursor phase or any mixture thereof from a suspension on the top layer of said cleansed organic PE film, so that at least one nanometer to micron-sized layer comprising calcium containing matrix is obtained;
iv. removing the residual calcium containing solution by washing;
v. adsorbing polyelectrolytes on top of said calcium phosphate layer; and,
vi. optionally repeating said procedure until a SAPF comprising a plurality of N organic PE films alternating with M layers of inorganic particles is formed, wherein N≧1 and M≧1.

The obtained SAPF is then immersed into a calcifying solution for a specified time, until the desired crystalline precipitate is formed, and the growth of crystalline calcium phosphate through said organic polyelectrolyte films is induced and sustained. The calcifying solution comprises a solution containing calcium and phosphate ions and/or any other ions in an effective amount necessary for a particular purpose. Said solution is supersaturated to the desired crystalline phase, but metastable, meaning that no precipitate should form without the presence of a “seeding” substrate.

After the desired crystalline calcium phosphate or other inorganic particles have been formed, the residual calcifying solution is removed by washing and optionally; the coated samples are dried and prepared for further use.

Coatings, prepared according to the aforementioned methods are deposited onto any suitable substrate, preferably to substrates at least partially made of materials selected from composite materials, glass ceramics, polymer, metal, and metal alloys, and/or built directly on top of the surfaces. A suitable metal will be chosen from the group of bioinert metals or metal alloys, which are deemed suitable for metal implants with load-bearing applications. Such are titanium, titanium based alloys, (Ti-6A1-4V and others), stainless steel, tantalum, zirconium, alloys based thereon, etc.

It is in the scope of the present invention wherein the aforementioned compositions are forming, coating, filling, replacing or reinforcing implants. It is also in the scope of the present invention wherein the aforementioned term ‘implant’ is denoted in a non limiting manner for any biodegradable or nondegradable implants; prosthetic components; bone substitute materials, artificial bone materials, glues, sealants or cements; orthopedic or other surgical inserts; dental implants, dental prosthesis or any combination thereof. It is also in the scope of the present invention wherein said implant is characterized by any suitable shape or size in the manner that it is adapted to be inserted into or onto humans or animals body.

It is also in the scope of the present invention wherein the said implants provided according to the present invention can be used for drug delivery, controlled release or sustained release of minerals or salts; organic substances; medicaments; drugs; cytokines, hormones, regulators of the bone metabolism and growth; antibiotics, biocide and bactericide drugs or peptides, DNA, RNA, amino acids, peptides, proteins, enzymes, cells, viruses and/or a combination thereof.

### EXAMPLE 1

Buildup of SAPF and Adsorption of Amorphous Calcium Phosphate on Glass Plates.

Materials and methods: Poly(L-lysine) (PLL, MW 3.26×10⁴ Da), poly(L-glutamic acid) (PGA, MW 7.2×10⁴ Da), tris(hydroxymethyl) aminomethane (TRIS), 2-(N-morpholino) ethanesulfonic acid (MES), N-2-Hydroxyethylpiperazine-N′-2-ethanesulfonic acid (HEPES), and NaCl from Sigma, and ultrapure water, UPW (Milli Q-plus system, Millipore or Barnstead) were used. MES/TRIS/NaCl or HEPES buffer solutions of pH 7.4 were prepared as follows: MES/TRIS/NaCl buffer: 25 mmol of MES, 25 mmol of TRIS and 100 mmol of NaCl were dissolved in 1 liter of UPW. HEPES/NaCl buffer: 25 mmol of HEPES and 150 mmol NaCl were dissolved in 1 l of UPW. Polyelectrolyte solutions were always freshly prepared by direct dissolution of the respective adequate weights in filtered buffer solutions. Suspensions of ACP were freshly prepared for each experiment by rapidly mixing equal volumes of 3, 5 or 10 mmolar equimolar solutions of calcium chloride and sodium phosphate in UPW or in HEPES buffer. The sodium phosphate solutions were adjusted to pH 7.4 before mixing.

The deposition of (PLL/PGA)ᵢ (wherein i is the number of layer pairs) and subsequent deposition of ACP was demonstrated by Optical Waveguide Lightmode Spectroscopy, denoted hereto in the term ‘OWLS’, and/or visualized by scanning electron microscopy, denoted hereto in the term ‘SEM’.

The optical waveguide lightmode spectroscopy technique (i.e., OWLS) is an optical technique, which gives information on the quantity, thickness and effective refractive index of an adsorbed layer onto a planar waveguide. OWLS is based on the effective refractive index change of a waveguide during the adsorption processes. Laser light which is incoupled into the waveguide is recorded and is proportional to the adsorbed amount of material. PLL/PGA PE films were built in-situ in the OWLS cell. In order to perform measurements, the system was rinsed with buffer, to remove all impurities. After the buffer flow was stopped, 100 μL of poly-L-lysine solution were manually injected into the cell through the injection port. After 12-15 min, sufficient to reach a plateau, the buffer flow was restarted for 12-15 min to rinse the excess material from the cell. In the same way the alternate adsorption of polyanions and polycations was continued and, progressively, (PLL/PGA)ᵢ multilayers were deposited. The film build-up was stopped for i=6 to obtain a negatively charged surface and for i=5 plus PLL to obtain a positive surface. After completion of the respective multilayer, 300 μl of a freshly prepared suspension of ACP were injected several times. Before the addition of ACP the system was rinsed for about 15 min with UPW, adjusted to pH 7.4 (FIGS. 1a,b) or HEPES buffer, pH 7.4 (FIG. 1c). For SEM (JOEL JSM-840 Scanning Microscope) analysis samples were prepared separately on glass plates, wherein the procedure was the same as in the OWLS experiment (see above). After deposition of ACP all plates were washed with UPW, dried in a stream of nitrogen and kept at 4° C. until analysis.

Reference is made now to FIG. 1A and FIG. 1B, showing the data recorded by OWLS for the build-up of SAPF from MES/TRIS buffer, ending with a positive (a) and a negative (b) film respectively. Also shown is the subsequent adsorption of ACP particles thereon. The continuous increase of the refractive index in the transverse electric mode, N(TE), shows the alternate deposition of the polyelectrolytes. One can observe the step by step layering of the polyelectrolyte films, each time followed by a plateau during the rinsing step. Rinsing of the SAPF with UPW before introducing the ACP suspension causes a slight decrease of the refractive index, followed by an increase, indicating the adsorption of ACP particles. FIG. 1c shows the build-up of (PLL/PGA)₆ from HEPES buffer and the subsequent deposition of ACP particles. No decrease in the refractive index is apparent because there was no change in the medium before and during the introduction of ACP.

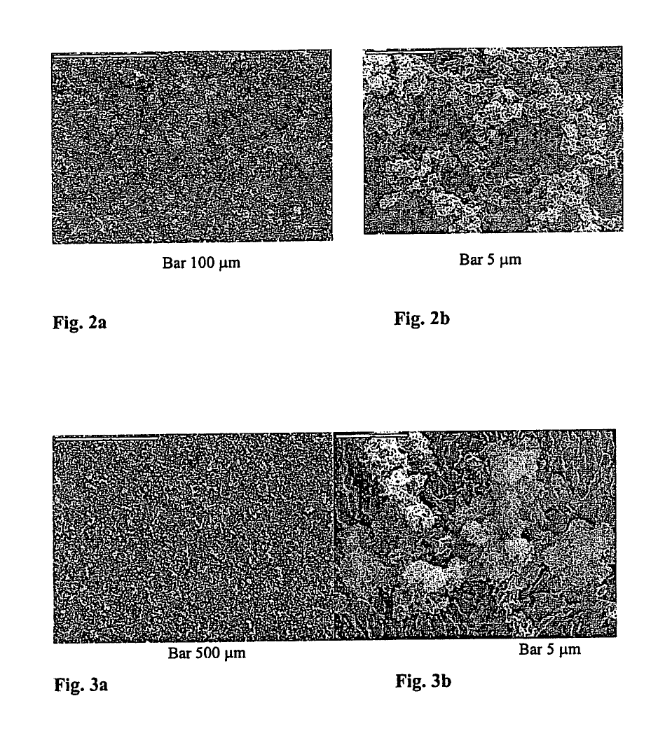

Reference is made now to FIG. 2, presenting SEM micrographs of aggregated ACP particles deposited on glass plates, coated with (PLL/PGA)₁₅ SAPF (two different magnifications). Similar SEM micrographs were obtained when ACP was deposited on (PLL/PGA)₁₄PLL. It is obvious from the above results, that ACP could be adsorbed on both positively and negatively charged multilayer films.

### EXAMPLE 2

Build-Up of SAPF on Ti Plates and Deposition of ACP Particles Upon Them.

Materials and Methods: Pure titanium plates, were received courtesy of Dentaurum, J. P. Winkelstroeter AG, Germany (Titanium ASTM grade 4, diameter 15 mm, thickness 1.5 mm, machine polished to a surface roughness Ra 0.4 μm, Rmax 3.0 μm and cleaned in perchloroethylene) and courtesy of SAMO S.p.A., Italy (Titanium ASTM grade 2, 1×1 cm, thickness 1.5 mm, chemically etched by SAMO). Before coating, plates were sonicated subsequently in acetone (p.a.), ethanol (p.a.) and three times in UPW. Each procedure lasted 10-15 min. XRD spectra of the bare plates showed only peaks characteristic of Ti.

Material A: (PLL/PGA)ᵢ and (PLL/PGA)ᵢPLL (i=9 or 14) multilayers were deposited as described in Example 1, using 1 ml of the respective solutions of PLL, PGA and HEPES/NaCl buffer pH 7.4. The plates with adsorbed multilayers were washed with buffer before depositing ACP particles. Plates were dipped three times into suspensions of ACP prepared in HEPES buffer as described in example 1, using 10 mmolar equimolar solutions of calcium chloride and sodium phosphate. After deposition the ACP plates were washed with buffer.

Material B was prepared by depositing [(PLL/PGA)₅-ACP]ᵢ or [(PLL/PGA)₄PLL-ACP]ᵢ (i=1-4) on material A. The preparation could be demonstrated by OWLS (not shown).

After preparation of materials A and B all plates were washed with buffer, dried in a stream of nitrogen and kept at 4° C. until further analysis. Samples thus prepared were observed by scanning electron microscopy (JOEL JSM-840 Scanning Microscope) and analyzed by powder X-ray diffraction.

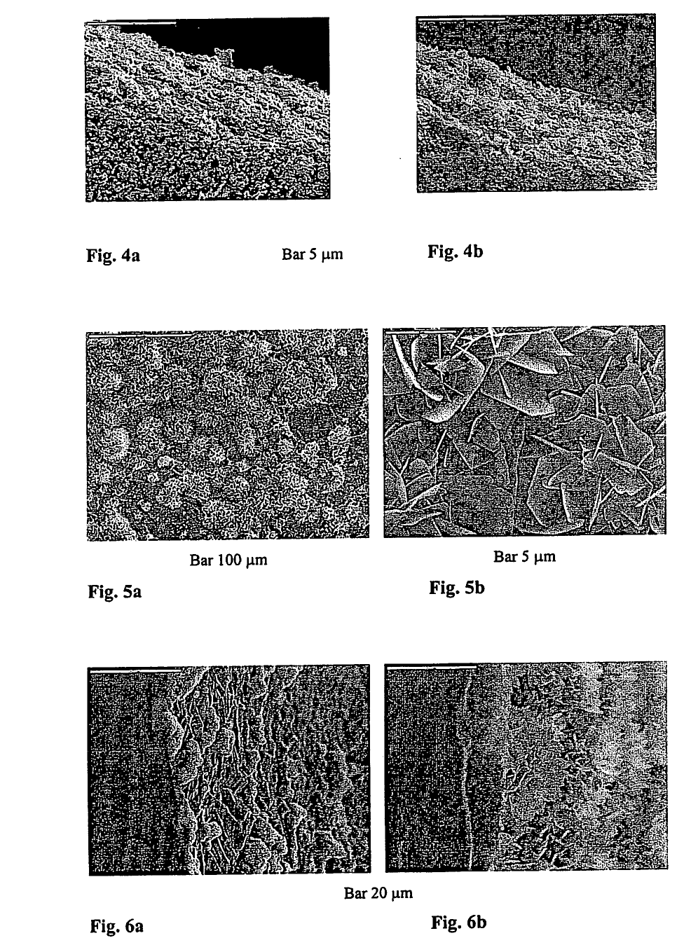

Reference is made now to FIGS. 3 and 4 showing four scanning electron micrographs of aggregated ACP particles in materials A and B. As expected, surface coverage is denser in material B. XRD diffraction patterns showed only Ti peaks, indicating that the deposited calcium phosphate phase is indeed amorphous.

### EXAMPLE 3

Coatings C and D Obtained by Build-Up of SAPF+ACP on Ti Plates and In-Situ Growth of OCP Crystals.

Coatings C and D: Materials A and B, respectively, were prepared on Ti plates as described in Example 2. Thus prepared plates were immersed into a calcifying solution (2.8 mmol/1 CaCl₂, 2 mmol/1 Na₂HPO₄, 25 mmol/1 HEPES, 150 mmol/1 NaCl, pH 7.4) for 48 hours. By this procedure material A converted into coating C, whereas material B gave coating D. After the crystallizing procedure all plates were washed with buffer, dried in a stream of nitrogen and kept at 4° C. until further analysis by X-ray powder diffraction and SEM. The adhesive tape test was conducted according to ASTM D 3359-92a and the tested specimens were observed with SEM.

Reference is made now to FIG. 5 showing SEM micrographs of coating C. Large, well developed, plate-like crystals, oriented perpendicular to the substrate were obtained. Apparently, the crystals grew from the previously deposited aggregated ACP particles (see FIG. 3b, Example 2).

Reference is made now to FIG. 6, presenting side views of SEM micrographs of: (a) coating C and (b) coating D. As in Example 2, the surface coverage improved with the number of SAPF's and ACP deposition steps, i.e. surface coverage of the plates was better in the case of coating D as compared to coating C. It is also apparent that the crystals were not deposited in layers, but grew through the whole PE multilayer, thus comprising a real organic-inorganic composite system. The XRD pattern of the multilayered coating (not shown) clearly shows the two small-angle lines, characteristic of OCP.

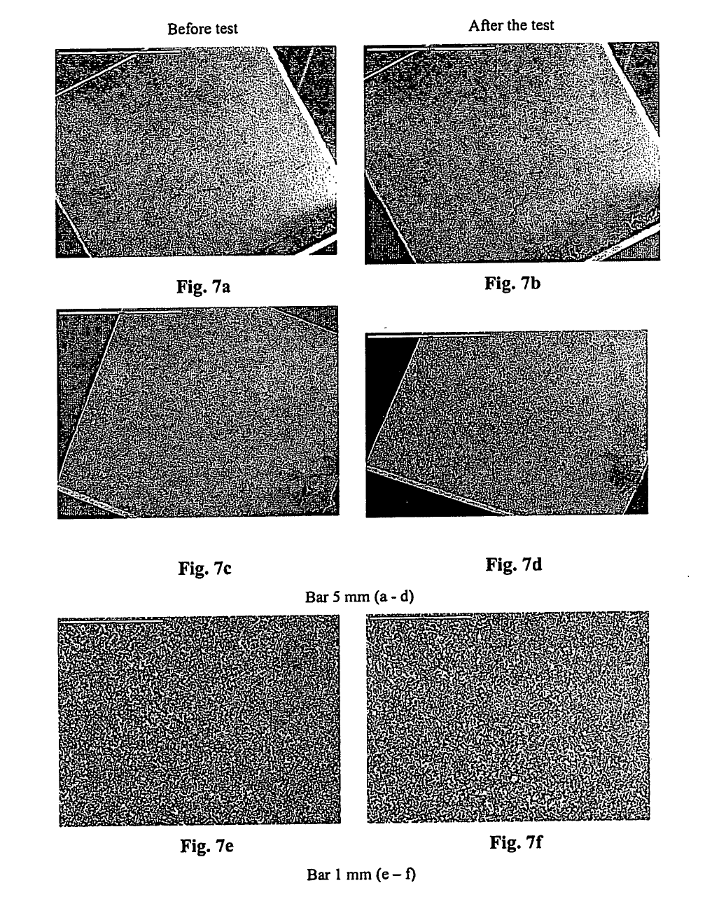

Reference is made now to FIG. 7, presenting the results of the adhesive tape test, showing that most of the coatings (including the crystals, FIGS. 7 e, f) remained intact on the Ti plates, indicating that the bonding between the plates and coatings C and D is good.

### EXAMPLE 4

Coatings C+ and D+, Prepared with PE ML as Top Layer

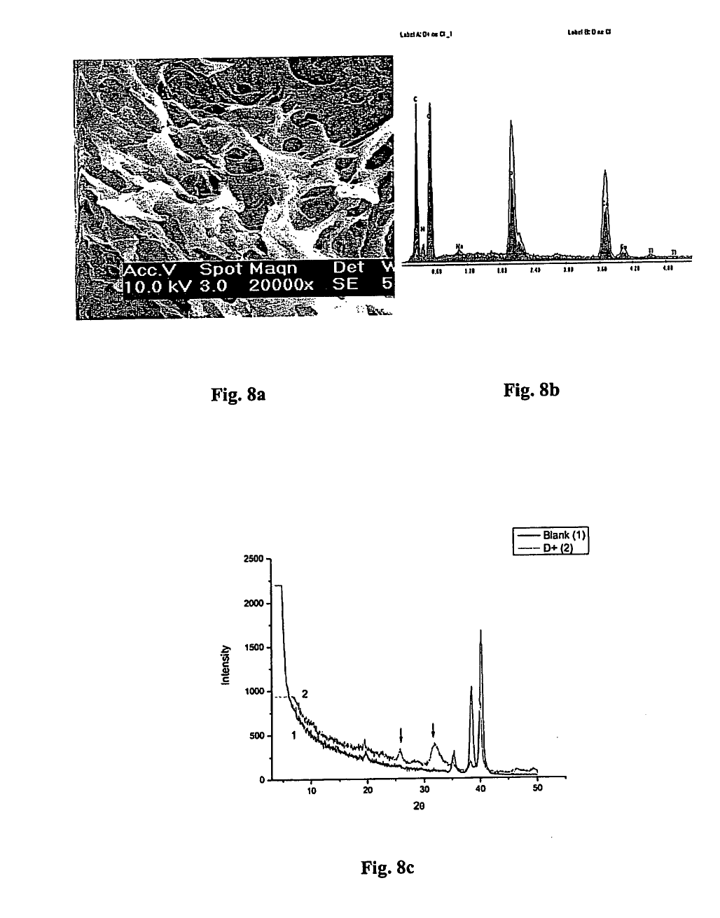

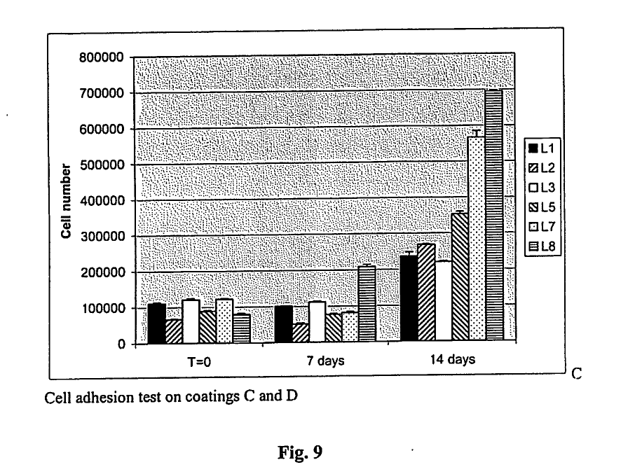

Materials A+ and B+were prepared similarly as materials A and B (see example 2) with the difference that an additional PLL/PGA multilayer was deposited on top of the last layer of ACP particles. Reference is now made to FIGS. 8a and 8b representing SEM micrographs and an EDX spectrum of coating D+. Clearly the coating is a nanocrystalline composite. Individual crystals are not apparent, but the EDX spectrum shows both C, O and N peaks from the organic phase and Ca and P peaks from the inorganic crystals. Reference is now made to FIG. 9c, representing a thin layer XRD spectrum with peaks corresponding to apatite, showing the inorganic phase is present as nanocrystalline apatite.

Reference is made now to FIG. 9, presenting a cell culture experiment. The cells were human primary osteoblast and were deposited onto six different substrates. Three substrates, L1, L2 and L8, respectively, are the reference standards and the golden standard for osteoblast cell adhesion and proliferation. The cell proliferation obtained after 14 days proved the bioactivity of organic-inorganic nanocomposites C+(L5) and D+(L7) as compared to bare titanium (L1) and titanium coated only by SAPF (L3), or inorganic particles (L2).

It is another objective of the present invention to provide a plurality of novel methods for the construction of SAPF materials. The methods to deposit PE layers and inorganic particles are not restricted to injection of solutions onto the substrate, i.e., injection coating, but include also spraying and dipping methods. The surface to be coated can be any surface as defined above. Sequentially depositing on a surface alternating layers of polyelectrolytes may be accomplished in a number of ways. The depositing process generally involves coating and rinsing steps. One coating process involves solely dip-coating and dip-rinsing steps. Another coating process involves solely spray-coating and spray-rinsing steps. However, a number of alternatives, involving various combinations of spray-, dip-, injection-coating and/or rinsing steps, may be designed by a person having ordinary skills in the art. According to one preferred embodiment of the present invention, the aforementioned method is provided by means of depositing the PE calcified films.

Moreover, and according to yet another embodiment of the present invention, in situ growing films of crystalline calcium phosphate phases onto biocompatible SAPF have been provided. The nature of the calcium phosphate particles, grown in situ on and within the multilayer is strictly controlled, e.g., by controlling the experimental conditions and the time of exposure of the coated substrate to the calcifying solution. Any of the following mineral phases, octacalcium phosphate, calcium deficient apatite, carbonate apatite, hydroxyapatite or mixtures thereof may grow in situ under mild, close to physiological experimental conditions, e.g., low reactant concentrations, room temperature, approx. neutral pH etc. The present invention also provided to produce alternating layers, containing different calcium phosphate phases within the multilayer or embedding previously prepared mineral with specially designed characteristics.

FIGS. 5, 6 and 8 show that the organic-inorganic nanocomposites, containing calcium phosphate crystals, grown as described in the present invention, are porous characterized by a relatively large surface area. The sizes of the crystals shown in FIG. 5b were between 1 and 2 μm, not exceeding 2 μm, but the sizes of crystals grown within a coating topped by a PLL/PGA multilayer were even smaller, in the nanometer size range. The resulting coating may be of any desired thickness and therefore should have the necessary strength and toughness, but also the porosity necessary for bioactive bone implants. By intergrowth (FIGS. 6a and 6b) with the SAPF the calcium phosphate layer fixed the nanocomposite coating to the underlying surface, so that very good adhesion was obtained (see FIG. 7).

Finally, in the examples given above, the deposition of coatings proposed in this invention on two different substrates: glass, and metal, e.g., titanium, was demonstrated. In fact, such coatings can be deposited on any hydrophilic substrate regardless of size, shape and topology. The methods employed to produce the coatings are environmental friendly, cost effective, energy saving and simple to perform.

While this invention has been described in terms of some specific examples, many modifications and variations are possible. It is therefore understood that within the scope of the appended claims, the invention may be realized otherwise than as specifically described.

1-15. (canceled)

16. A method of preparing an organic-inorganic composite comprising a plurality of organic polyelectrolyte films, interspersed with inorganic bioactive particles growing through said organic films, comprising the steps of:
a. adsorbing polyelectrolytes on top of a surface so that at least one polyelectrolyte film is obtained;
b. washing the obtained film in the manner that residual polyelectrolytes are removed;
c. depositing nanosized to micron-sized particles comprising calcium phosphate on top of said polyelectrolyte films, so that at least one layer comprising bioactive inorganic material is formed;
d. washing the obtained layer in the manner that residual calcium containing solution is removed; and
e. immersing the material into a calcifying solution in the manner that the growth of crystalline calcium phosphate through said organic polyelectrolyte films is induced and sustained.

17. A method according to claim 16 comprising the steps of:
a. adsorbing polyelectrolytes on top of a surface so that at least one polyelectrolyte film is obtained;
b. washing the obtained film in the manner that residual polyelectrolytes are removed;
c. depositing nanosized to micron-sized particles comprising calcium phosphate on top of said polyelectrolyte film, so that at least one layer comprising bioactive inorganic material is formed;
d. washing the obtained layer in the manner that residual calcium containing solution is removed; and
e. adsorbing polyelectrolytes on top of said calcium phosphate layer;
f. repeating steps b. to e. at least once; and
g. immersing the obtained multilayer material into a calcifying solution in the manner that in situ growth of calcium phosphate crystals is induced and sustained through said polyelectrolyte films.

18. A method according to claim 16, wherein each of said polyelectrolyte films comprises at least one polycationic and at least one polyanionic polymer.

19. A method according to claim 16, wherein said polyelectrolyte comprises a material selected from the group consisting of polyaminoacids, polynucleotides, proteins, and polysaccharides.

20. A method according to claim 16, wherein said polyelectrolyte is selected from the group consisting of poly-arginine, poly-lysine, poly-glutamic acid, poly-aspartic acid, polyinosinic acid, polycytidylic acid, polythymidylic acid, polyguanylic acid, silk, amelogenin, albumin, sialoprotein, osteocalcin, phosphophoryn, phosvitin, fibrinogen, fibronectin, collagen, elastin, lectines, phosphoproteins, heparan, chondroitin, chondroitin sulfate, proteoglycans, heparin, hyaluronic acid, glucosaminoglycan, polygalacturonic acid, chitosan, alginate, lipopolysacharides, polyphosphonates, polyphosphates, derivatives thereof, and a mixture thereof.

21. A method according to claim 16, wherein said organic polyelectrolyte films further comprise a component selected from the group consisting of poly-leucine, poly-serine, poly-hydroxyproline, poly(lactide), poly(styrene), poly(ethylene), poly(oxyethylene), poly(acrylic)acid, poly(methacrylic)acid, poly(maleimide), dextrin, cyclodextrin, agarose, and cellulose.

22. A method according to claim 16, wherein said inorganic layer of bioactive particles comprises crystalline calcium phosphates.

23. A method according to claim 22, wherein said crystalline calcium phosphates comprise calcium hydrogen phosphate, octacalcium phosphate, tri-calcium phosphate, calcium deficient apatite, carbonated apatite, stoichiometric hydroxyapatite, crystalline calcium phosphates containing foreign ions, crystalline calcium phosphates containing cytokines, crystalline calcium phosphates containing peptides, their derivatives or any combination thereof.

24. An organic-inorganic composite prepared by the method of claim 16, comprising a plurality of organic polyelectrolyte films interspersed with nanometer to micron-sized inorganic bioactive particles.

25. A composite according to claim 24, wherein said bioactive particles comprise amorphous or crystalline matter.

26. A composite according to claim 24, wherein each of said polyelectrolyte films comprises at least one polycationic and at least one polyanionic polymer.

27. A composite according to claim 26, wherein said polymers are selected from the group consisting of poly-arginine, poly-lysine, poly-glutamic acid, poly-aspartic acid, polyaminoacids, polyinosinic acid, polycytidylic acid, polythymidylic acid, polyguanylic acid, polygalacturonic acid, silk, amelogenin, albumin, sialoprotein, osteocalcin, phosphophoryn, phosvitin, polyphosphonates, polyphosphates, phosphoproteins, lectines, lipopolysacharides, fibrinogen, fibronectin, heparin, chitosan, hyaluronic acid, alginate, collagen, glucosaminoglycan, heparan, chondroitin, chondroitin sulfate, elastin, proteoglycan, derivatives thereof, and a mixture thereof.

28. A composite according to claim 24, wherein said bioactive particles comprise crystalline calcium phosphates.

29. A composite according to claim 28, wherein said crystalline calcium phosphates comprise calcium hydrogen phosphate, octacalcium phosphate, tri-calcium phosphate, calcium deficient apatite, carbonated apatite, stoichiometric hydroxyapatite, crystalline calcium phosphates containing foreign ions, crystalline calcium phosphates containing cytokines, crystalline calcium phosphates containing peptides, their derivatives or any combination thereof.

30. Bioactive nanocomposite coatings comprising a composite according to claim 24.

31. Implants, comprising a composite according to claim 24.

32. Implants at least partially coated by a composite according to claim 24 in the manner that a significant portion of said implants are coated by a bioactive nanocomposite.

33. An implant according to claim 31, at least partially made of materials selected from composite materials, glass ceramics, polymer, metal, metal alloys, or any combination thereof.

34. An implant according to claim 33, wherein the metal or metal alloy comprise titanium, titanium based alloys, stainless steel, tantalum, zirconium, nickel, iridium, niobium, palladium, or nickel-titanium.
